(19) United States
(12) Patent Application Publication
(10) Pub. No.: US 2006/0194847 A1
(43) Pub. Date: Aug. 31, 2006

(54) Novel 6-substituted 2-aminopyridine derivatives

(76) Inventors: David Cheshire, Leicestershire (GB); Stephen Connolly, Leicestershire (GB); Timothy Luker, Leicestershire (GB)

Correspondence Address: FISH & RICHARDSON P.C., P.O BOX 1022, MINNEAPOLIS, MN, 55440-1022, US

(21) Appl. No.: 10/551,494

(22) PCT Filed: Mar. 30, 2004
(86) PCT No.: PCT/SE04/00487

§ 371 Date: Sep. 29, 2005

(30) Foreign Application Priority Data
Mar. 31, 2003 (SE) 0300907.3

(51) Int. Cl. C07D 213/75; A61K 31/44

(52) U.S. Cl. 514/352; 546/309

(57) ABSTRACT

There are provided novel compounds of formula (I) wherein R¹, R², R³, R⁴, R⁵, R⁶, R⁷, R⁸, R⁹, L¹, L², L³ and Q are are as defined in the specification, and pharmaceutically acceptable salts thereof; together with processes for their preparation, compositions containing them and their use in therapy. The compounds are inhibitors of nitric oxide synthase and are thereby particularly useful in the treatment or prophylaxis of inflammatory disease and pain.

### FIELD OF THE INVENTION

The present invention relates to novel 6-substituted 2-aminopyridine derivatives, processes for their preparation, compositions containing them and their use in therapy.

### BACKGROUND OF THE INVENTION

Nitric oxide is produced in mammalian cells from L-arginine by the action of specific nitric oxide synthases (NOSs). These enzymes fall into two distinct classes—constitutive NOS (cNOS) and inducible NOS (iNOS). At the present time, two constitutive NOSs and one inducible NOS have been identified. Of the constitutive NOSs, an endothelial enzyme (ecNOS) is involved with smooth muscle relaxation and the regulation of blood pressure and blood flow, whereas the neuronal enzyme (ncNOS) serves as a neurotransmitter and appears to be involved in the regulation of various biological functions such as cerebral ischaemia. Inducible NOS has been particularly implicated in the pathogenesis of inflammatory diseases. Regulation of these enzymes should therefore offer considerable potential in the treatment of a wide variety of disease states (J. E. Macdonald, Ann. Rep. Med Chem., 1996, 31, 221-230).

Considerable effort has been expended in efforts to identify compounds that act as specific inhibitors of one or more isoforms of the enzyme nitric oxide synthase. The use of such compounds in therapy has also been widely claimed.

### DISCLOSURE OF THE INVENTION

According to the present invention, there is provided a compound of formula (I) 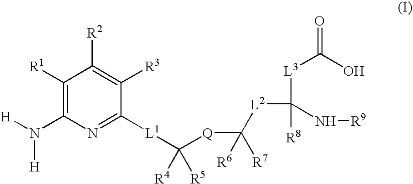
wherein

R¹, R² and R³ independently represent H, halogen, C1 to 4 alkyl, C1 to 4 alkoxy, CN, MeS(O)ₘ or NR¹⁰R¹¹; said alkyl group being optionally further substituted by OH or one or more halogen atoms;

L¹ and L² independently represent a bond or CR¹²R¹³ wherein R¹² and R¹³ independently represent H or C1 to 4 alkyl; said alkyl being optionally further substituted by OH, C1 to 2 alkoxy, CN or one or more halogen atoms;

L³ represents —CH₂— or a bond;

R⁴, R⁵, R⁶ and R⁷ independently represent H, C1 to 6 alkyl, Ar¹ or Ar¹—C1 to 4 alkyl;

or R⁴ and R⁵, or R⁶ and R⁷, may be joined together such that the group CR⁴R⁵ or the group CR⁶R⁷ represents a C3 to 6 cycloalkyl ring;

Q represents O, S(O)ₙ or NR¹⁶;

R¹⁶ represents H, C1 to 6 alkyl, C1 to 6 alkanoyl, C1 to 6 alkyl-SO₂—, C1 to 6 alkyl-O—CO—, Ar² or Ar²—CH₂—;

Ar¹ and Ar² independently represents phenyl or a 5- or 6-membered heteroaromatic ring containing one to three heteroatoms independently selected from O, S and N; said phenyl or heteroaromatic ring being optionally substituted by one or more substituents independently selected from halogen, CN, CF₃, C1 to 3 alkyl, C1 to 3 alkoxy, hydroxy, C1 to 3 thioalkoxy or NR¹⁴R¹⁵;

m and n independently represent an integer 0, 1 or 2;

R⁸ represents H or C1 to 4 alkyl; said alkyl being optionally further substituted by OH, C1 to 2 alkoxy, CN or one or more halogen atoms;

R⁹ represents H or C1 to 4 alkyl;

R¹⁰ and R¹¹ independently represent H, C1 to 2 alkyl, C1 to 2 alkanoyl or C1 to 2 alkylsulfonyl;

R¹⁴ and R¹⁵ independently represent H, C1 to 4 alkyl, C1 to 2 alkylsulfonyl or C1 to 4 alkanoyl; said alkyl being optionally further substituted by OH, C1 to 2 alkoxy, CN or one or more halogen atoms;

and pharmaceutically acceptable salts thereof.

Certain compounds of formula (I) are capable of existing in stereoisomeric forms. It will be understood that the invention encompasses all geometric and optical isomers of the compounds of formula (I) and mixtures thereof including racemates. Certain compounds of formula (I) are capable of existing in tautomeric forms. All such tautomers and mixtures thereof also form an aspect of the present invention.

In one embodiment, L³ represents a bond. In another embodiment, L¹ represents a bond or —CR¹²R¹³— wherein R¹² and R¹³ independently represent H or C1 to 4 alkyl. In another embodiment, L² represents a bond or —CR¹²R³— wherein R¹² and R¹³ independently represent H or C1 to 4 alkyl. In another embodiment, L¹ and L² each independently represent a bond or —CR¹²R¹³— wherein R¹² and R¹³ independently represent H or C1 to 4 alkyl and L³ represents a bond.

In one embodiment R² represents H or C1 to 4 alkyl-and R¹ and R³ each represent H. In one embodiment, R² represents CH₃.

In one embodiment, Q represents O.

In one embodiment, Q represents S.

In one embodiment, Q represents NR¹⁶ and R¹⁶ represents H or C1 to 6 alkyl.

In one embodiment, R⁴, R⁵, R⁶ and R⁷ each independently represent H or C1 to 4 alkyl.

The compounds of formula (I) and their pharmaceutically acceptable salts have the advantage that they are inhibitors of the enzyme nitric oxide synthase (NOS). In particular, the compounds of formula (I) and their pharmaceutically acceptable salts have the advantage that they are inhibitors of the inducible isoform of the enzyme nitric oxide synthase (iNOS).

The invention further provides a process for the preparation of compounds of formula (I) or a pharmaceutically acceptable salt, enantiomer or racemate thereof.

According to the invention there is also provided a compound of formula (I), or a pharmaceutically acceptable salt thereof for use as a medicament.

Another aspect of the invention provides the use of a compound of formula (I) or a pharmaceutically acceptable salt thereof, in the manufacture of a medicament, for the treatment or prophylaxis of diseases or conditions in which inhibition of nitric oxide synthase activity is beneficial.

A more particular aspect of the invention provides the use of a compound of formula (I) or a pharmaceutically acceptable salt thereof, in the manufacture of a medicament, for the treatment or prophylaxis of inflammatory disease.

According to the invention, there is also provided a method of treating, or reducing the risk of, diseases or conditions in which inhibition of nitric oxide synthase activity is beneficial which comprises administering to a person suffering from or at risk of, said disease or condition, a therapeutically effective amount of a compound of formula (I) or a pharmaceutically acceptable salt thereof.

More particularly, there is also provided a method of treating, or reducing the risk of, inflammatory disease in a person suffering from or at risk of, said disease, wherein the method comprises administering to the person a therapeutically effective amount of a compound of formula (I) or a pharmaceutically acceptable salt thereof.

The compounds of the present invention may also be used advantageously in combination with a second pharmaceutically active substance; particularly in combination with a cyclooxygenase inhibitor; more particularly in combination with a selective inhibitor of the inducible isoform of cyclooxygenase (COX-2). Thus, in a further aspect of the invention there is provided the use of a compound of formula (I) or a pharmaceutically acceptable salt thereof, in combination with a COX-2 inhibitor for the treatment of inflammation, inflammatory disease and inflammatory related disorders. And there is also provided a method of treating, or reducing the risk of, inflammation, inflammatory disease and inflammatory related disorders in a person suffering from or at risk of, said disease or condition, wherein the method comprises administering to the person a therapeutically effective amount of a compound of formula (I) or a pharmaceutically acceptable salt thereof in combination with a COX-2 inhibitor.

Particular compounds of the invention include:
S-[(6-amino-4-methyl-2-pyridinyl)methyl]-L-cysteine;
S-[2-(6-amino-4-methyl-2-pyridinyl)ethyl]-L-cysteine;
S-[(6-amino-4-methyl-2-pyridinyl)methyl]-L-homocysteine;
S-[(6-amino-4-methyl-2-pyridinyl)methyl]-2-methyl-L-cysteine;
(3R)—S-[(6-amino-4-methyl-2-pyridinyl)methyl]-3-methyl-L-cysteine;
O-[(6-amino-4-methyl-2-pyridinyl)methyl]-L-serine;
O-[(6-amino-4-methyl-2-pyridinyl)methyl]-D-serine;
3-[[(6-amino-4-methyl-2-pyridinyl)methyl](methylsulfonyl)amino]-L-alanine;
3-[[(6-amino-4-methyl-2-pyridinyl)methyl]amino]-L-alanine;
(3S)-S-[(6-amino-4-methyl-2-pyridinyl)methyl]-3-methyl-L-cysteine;
   and pharmaceutically acceptable salts thereof.

Unless otherwise indicated, the term “C1 to 6 alkyl” referred to herein denotes a straight or branched chain alkyl group having from 1 to 6 carbon atoms. Examples of such groups include methyl, ethyl, n-propyl, i-propyl, n-butyl, i-butyl, t-butyl, pentyl and hexyl. The terms “C1 to 2 alkyl”, “C1 to 3 alkyl” and “C1 to 4 alkyl” are to be interpreted analogously.

Unless otherwise indicated, the term “C1 to 4 alkoxy” referred to herein denotes a straight or branched chain alkoxy group having from 1 to 4 carbon atoms. Examples of such groups include methoxy, ethoxy, n-propoxy and i-propoxy. The terms “C1 to 2 alkoxy” and “C1to 3 alkoxy” are to be interpreted analogously.

Unless otherwise indicated, the term “C1 to 3 thioalkoxy” referred to herein denotes a straight or branched chain alkyl group having from 1 to 3 carbon atoms bonded to a sulphur atom. Examples of such groups include methylthio, ethylthio, n-propylthio and i-propylthio.

Unless otherwise indicated, the term “C3 to 6 cycloalkyl” referred to herein denotes a saturated carbocyclic ring having from 3 to 6 carbon atoms. Examples of such groups include cyclopropyl, cyclopentyl and cyclohexyl.

Unless otherwise indicated, the term “C1 to 6 alkanoyl” referred to herein denotes formyl or a straight or branched chain alkyl group having from 2 to 6 carbon atoms bonded to a carbonyl group. Examples of such groups include acetyl, n-propanoyl, i-propanoyl and butanoyl. The terms “C1 to 4 alkanoyl” and “C1 to 2 alkanoyl” are to be interpreted analogously.

Unless otherwise indicated, the term “halogen” referred to herein denotes fluoro, chloro, bromo and iodo.

Examples of a “C1 to 4 alkyl optionally further substituted by one or more halogen atoms” include CH₂F, CH₂Cl, CH₂Br, CHF₂, CF₃, CF₃CF₂, CF₃CH₂, CH₂FCH₂, CH₃CF₂ and CF₃CH₂CH₂.

Examples of a group “Ar¹—C1 to 4 alkyl'include Ar—CH₂—, Ar¹—CH₂CH₂— and Ar¹—CH(CH₃)—.

Examples of a 5- or 6-membered heteroaromatic ring containing one to three heteroatoms independently selected from O, S and N include furan, thiophene, thiazole, isoxazole, imidazole, triazole, thiadiazole, pyridine, pyrimidine and pyrazine.

Examples of a group “C1 to 6 alkyl-SO₂—” include methylsulphonyl, ethylsulphonyl and propylsulphonyl. The term “C1 to 2 alkylsulphonyl” denotes methylsulphonyl or ethylsulphonyl.

Examples of a group “C1 to 6 alkyl-O—CO—” include methoxycarbonyl, ethoxycarbonyl and propoxycarbonyl.

According to the invention, we further provide a process for the preparation of compounds of formula (I), or a pharmaceutically acceptable salt, enantiomer or racemate thereof which process [wherein variable groups are, unless otherwise specified, as defined in formula (I)] comprises:

(a) reaction of a compound of formula (II) 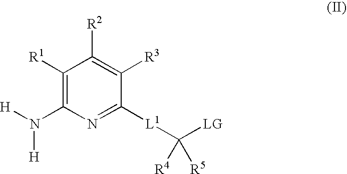
wherein LG represents a leaving group,

with a compound of formula (III) 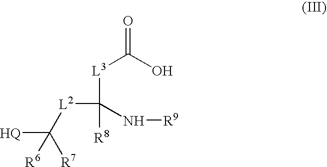
or

(b) reaction of a compound of formula (IV) 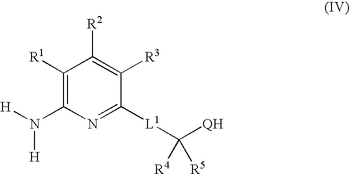
with a compound of formula (V) 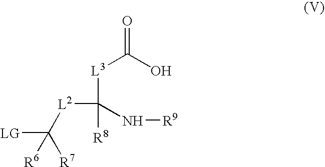
wherein LG is a leaving group; or

(c) when Q represents S, reacting a compound of formula (VI) 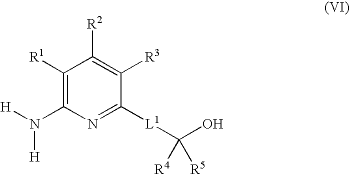
with a compound of formula (VII) 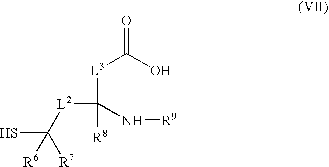
under Mitsunobu conditions;

and where desired or necessary converting the resultant compound of formula (I), or another salt thereof, into a pharmaceutically acceptable salt thereof; or converting one compound of formula (I) into another compound of formula (I); and where desired converting the resultant compound of formula (I) into an optical isomer thereof.

In processes (a) and (b), the reaction is performed by treating a nucleophile of formula (III) or (IV) with an electrophile of formula (II) or (V) respectively in an inert solvent. Suitable leaving groups LG include sulphonates and halides. The reaction is generally performed in the presence of a non-nucleophilic base such as sodium hydride, caesium carbonate, sodium bicarbonate or potassium hydroxide. Suitable organic solvents are those such as N,N-dimethylformamide, N-methyl-2-pyrrolidinone, tetrahydrofuran and dimethylsulfoxide. The reaction is generally conducted at a temperature between 0° C. and the boiling point of the solvent.

In process (c), the reactants (VI) and (VII) are coupled together in a suitable inert solvent such as tetrahydrofuran or dichloromethane using, for example, Mitsunobu conditions. Thus, for example, the reactants are treated with a phosphine derivative, an azo derivative and imidazole at a suitable temperature, generally between 0° C. and the boiling point of the solvent. Suitable phosphine derivatives include trimethylphosphine and tributylphosphine. Suitable azo derivatives include diethyl azodicarboxylate, diisopropyl azodicarboxylate, di-t-butyl azodicarboxylate and 1,1′-(azodicarbonyl)dipiperidine.

It will be apparent to a person skilled in the art that in the above processes it may be desirable or necessary to protect an amine, hydroxyl, carboxyl or other potentially reactive group. Suitable protecting groups and details of processes for adding and removing such groups may be found by reference to the standard text “Protective Groups in Organic Synthesis”, 3rd Edition (1999) by Greene and Wuts.

In one embodiment, amine groups are protected as carbamate derivatives, for example, as t-butyloxycarbamates. In another embodiment, the amino group of a 2-aminopyridine is protected as a 2,5-dimethylpyrrole. In another embodiment, carboxyl groups are protected as alkyl esters, for example, as methyl esters.

Specific examples of the use of protecting groups are given in the Examples section.

The present invention includes compounds of formula (I) in the form of salts, in particular acid addition salts. Suitable salts include those formed with both organic and inorganic acids. Such acid addition salts will normally be pharmaceutically acceptable although salts of non-pharmaceutically acceptable acids may be of utility in the preparation and purification of the compound in question. Thus, preferred salts include those formed from hydrochloric, hydrobromic, sulphuric, phosphoric, citric, tartaric, lactic, pyruvic, acetic, succinic, fumaric, maleic, methanesulphonic and benzenesulphonic acids.

Salts of compounds of formula (I) may be formed by reacting the free base, or a salt, enantiomer or racemate thereof, with one or more equivalents of the appropriate acid. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin.

Intermediate compounds may be used as such or in protected form. Protecting groups and details of processes for their removal may be found by reference to the standard text “Protective Groups in Organic Synthesis”, 3rd Edition (1999) by Greene and Wuts.

The compounds of the invention and intermediates thereto may be isolated from their reaction mixtures and, if necessary further purified, by using standard techniques.

The compounds of formula I may exist in enantiomeric forms. Therefore, all enantiomers, diastereomers, racemates and mixtures thereof are included within the scope of the invention. The various optical isomers may be isolated by separation of a racemic mixture of the compounds using conventional techniques, for example, fractional crystallisation, or HPLC.

Intermediate compounds may also exist in enantiomeric forms and may be used as purified enantiomers, diastereomers, racemates or mixtures.

The compounds of formula (I), and their pharmaceutically acceptable salts are useful because they possess pharmacological activity in animals. In particular, the compounds are active as inhibitors of the enzyme nitric oxide synthase. More particularly, they are inhibitors of the inducible isoform of the enzyme nitric oxide synthase and as such are predicted to be useful in therapy, for example, as anti-inflammatory agents. They may also have utility as inhibitors of the neuronal isoform of the enzyme nitric oxide synthase.

The compounds and their pharmaceutically acceptable salts are indicated for use in the treatment or prophylaxis of diseases or conditions in which synthesis or oversynthesis of nitric oxide synthase forms a contributory part. In particular, the compounds are indicated for use in the treatment of inflammatory conditions in mammals including man.

Conditions that may be specifically mentioned are:
osteoarthritis, rheumatoid arthritis, rheumatoid spondylitis, gouty arthritis and other arthritic conditions, inflamed joints;
eczema, psoriasis, dermatitis or other inflammatory skin conditions such as sunburn;
inflammatory eye conditions including uveitis, glaucoma and conjunctivitis;
lung disorders in which inflammation is involved, for example, asthma, bronchitis, chronic obstructive pulmonary disease, pigeon fancier's disease, farmer's lung, acute respiratory distress syndrome;
bacteraemia, endotoxaemia (septic shock), aphthous ulcers, gingivitis, pyresis, pain, meningitis and pancreatitis;
conditions of the gastrointestinal tract including inflammatory bowel disease, Crohn's disease, atrophic gastritis, gastritis varialoforme, ulcerative colitis, coeliac disease, regional ileitis, peptic ulceration, irritable bowel syndrome, reflux oesophagitis, damage to the gastrointestinal tract resulting from infections by, for example, Helicobacter pylori, or from treatments with non-steroidal anti-inflammatory drugs;
   and other conditions associated with inflammation.

The compounds will also be useful in the treatment and alleviation of acute pain or persistent inflammatory pain or neuropathic pain or pain of a central origin.

We are particularly interested in the conditions inflammatory bowel disease, rheumatoid arthritis, osteoarthritis, chronic obstructive pulmonary disease and pain.

The compounds of formula (I) and their pharmaceutically acceptable salts may also be useful in the treatment or prophylaxis of diseases or conditions in addition to those mentioned above. For example, the compounds may be useful in the treatment of atherosclerosis, cystic fibrosis, hypotension associated with septic and/or toxic shock, in the treatment of dysfunction of the immune system, as an adjuvant to short-term immunosuppression in organ transplant therapy, in the control of onset of diabetes, in the maintenance of pancreatic function in diabetes, in the treatment of vascular complications associated with diabetes and in co-therapy with cytokines, for example TNF or interleukins.

The compounds of formula (I) may also be useful in the treatment of hypoxia, for example in cases of cardiac arrest and stroke, neurodegenerative disorders including nerve degeneration is and/or nerve necrosis in disorders such as ischaemia, hypoxia, hypoglycaemia, epilepsy, and in external wounds (such as spinal cord and head injury), hyperbaric oxygen convulsions and toxicity, dementia, for example pre-senile dementia, Alzheimer's disease and AIDS-related dementia, Sydenham's chorea, Parkinson's disease, Tourette's syndrome, Huntington's disease, amyotrophic lateral sclerosis, multiple sclerosis, muscular dystrophy, Korsakoffs disease, imbecility relating to a cerebral vessel disorder, sleeping disorders, schizophrenia, depression, pain, autism, seasonal affective disorder, jet-lag, depression or other symptoms associated with premenstrual syndrome (PMS), anxiety and septic shock. Compounds of formula (I) may also be expected to show activity in the prevention and reversal of drug addiction or tolerance such as tolerance to opiates and diazepines, treatment of drug addiction, treatment of migraine and other vascular headaches, neurogenic inflammation, in the treatment of gastrointestinal motility disorders, cancer and in the induction of labour.

We are particularly interested in the conditions stroke, Alzheimer's disease, Parkinson's disease, multiple sclerosis, schizophrenia, migraine, cancer, septic shock and pain.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above mentioned therapeutic indications, the dosage administered will, of course, vary with the compound employed, the mode of administration and the treatment desired. However, in general, satisfactory results are obtained when the compounds are administered at a dosage of the solid form of between 1 mg and 2000 mg per day.

The compounds of formula (I), and pharmaceutically acceptable derivatives thereof, may be used on their own, or in the form of appropriate pharmaceutical compositions in which the compound or derivative is in admixture with a pharmaceutically acceptable adjuvant, diluent or carrier. Administration may be by, but is not limited to, enteral (including oral, sublingual or rectal), intranasal, inhalation, intravenous, topical or other parenteral routes. Conventional procedures for the selection and preparation of suitable pharmaceutical formulations are described in, for example, “Pharmaceuticals—The Science of Dosage Form Designs”, M. E. Aulton, Churchill Livingstone, 1988. The pharmaceutical composition preferably comprises less than 80% and more preferably less than 50% of a compound of formula (I), or a pharmaceutically acceptable salt thereof.

According to the invention, we further provide a pharmaceutical composition comprising a compound of formula (I), or a pharmaceutically acceptable salt thereof, in admixture with a pharmaceutically acceptable adjuvant, diluent or carrier.

There is also provided a process for the preparation of such a pharmaceutical composition which comprises mixing the ingredients.

The compounds of formula (I), and pharmaceutically acceptable derivatives thereof, may also be advantageously used in combination with one of the following therapies: NSAIDS, COX-2 inhibitors, Paracetamol, Tramadol, Corticosteroids, Glucosamine, Doxycyclin, Pralnacasan, MMP inhibitors or Coll-3 inhibitors. The compound of formula (I) and the combination therapy may either be formulated together within the same pharmaceutical composition for administration in a single dosage unit, or each component may be individually formulated such that separate dosages may be administered either simultaneously or sequentially.

The invention is illustrated, but in no way limited, by the following examples:

The following abbreviations are used:
DMF N,N-Dimethylformamide;
THF Tetrahydrofuran;
DCM Dichloromethane.

Unless otherwise indicated, organic solutions were dried over anhydrous sodium sulphate.

### EXAMPLE 1

### S-[(6-Amino-4-methyl-2-pyridinyl)methyl]-L-cysteine diacetate

### a) 2-(Chloromethyl)-6-(2,5-dimethyl-1H-pyrrol-1-yl)-4-methyl-pyridine

To a solution of n-butyl lithium (1.6M solution in hexanes, 6.6 ml) at −20° C. was added diethyl ether (13 ml) followed by a solution of 2-(2,5-dimethyl-1H-pyrrol-1-yl)-4,6-dimethyl-pyridine (Bioorganic and Medicinal Chemistry Letters, 2000, 10, 1975) (2 g) in diethyl ether (13 ml) dropwise, and the solution warmed to 0° C. over 1 h. After cooling to −78° C., THF (7 ml) was added, and the solution transferred by cannula into a solution of hexachloroethane (3.08 g) in THF (7 ml). The mixture was stirred for 30 min and then warmed to room temperature. Ethyl acetate and aqueous ammonium chloride solution were added and the organic layer was separated, dried and the solvent was removed in vacuo. The residue was purified by chromatography (silica, 0-5% diethyl ether/DCM as eluent) to give the sub-titled compound (2 g) as an oil.

MS APCI+ve ^{m}/z 235 ([M+H]⁺).

¹H NMR 400 MHz (CDCl₃) 7.31 (1H, s), 6.98 (1H, s), 5.89 (2H, s), 2.65 (2H, s), 2.45 (3H, s), 2.13 (6H, s).

### b) N-[(1,1-Dimethylethoxy)carbonyl]-S-[[6-(2,5-dimethyl-1H-pyrrol-1-yl)4-methyl-2-pyridinyl]methyl]-L-cysteine, methyl ester

To a solution of N-(tert-butoxycarbonyl)-L-cysteine methyl ester (0.40 g) in DMF (4 ml) was added sodium hydride (60% dispersion in mineral oil, 58 mg) and the reaction stirred for 1 h. A solution of the product from step (a) (0.40 g) in diethyl ether (4 ml) was added dropwise and the mixture stirred for 16 h. Ethyl acetate and water were added and the organic layer separated, then washed with water (5×), brine, dried and the solvent was removed in vacuo. The residue was purified by chromatography (silica, 0-8% diethyl ether/DCM as eluent) to give the sub-titled compound (0.42 g) as a colourless oil.

¹H NMR 400 MHz (CDCl₃) 7.18 (1H, s), 6.92 (1H, s), 5.87 (2H, s), 5.41 (1H, m), 4.53 (1H, m), 3.83 (2H, s), 3.72 (3H, s), 2.99 (1H, dd), 2.90 (1H, dd), 2.42 (3H, s), 2.11 (6H, s), 1.44 (9H, s).

### c) S-[(6-Amino-4-methyl-2-pyridinyl)methyl]-N-[(1,1-dimethylethoxy)carbonyl]-L-cysteine, methyl ester

To a solution of the product from step (b) (0.41 g) in ethanol (2.8 ml) and water (1.1 ml) was added hydroxylamine hydrochloride (0.30 g) and potassium hydroxide (0.17 g) and the reaction heated at 95° C. for 16 h. Water was added and the aqueous layer extracted with DCM (3×). The combined organics were dried and the solvent was removed in vacuo. The residue was purified by chromatography (silica, 0-5% methanol/DCM as eluent) to give the sub-titled compound (140 mg) as an orange oil.

MS APCI+ve ^{m}/z 356 ([M+H]⁺).

### d) S-[(6-Amino-4-methyl-2-pyridinyl)methyl]-L-cysteine diacetate

The compound from step (c) was treated with 5M aqueous HCl (6 ml) and heated at 100° C for 20 min. The mixture was concentrated and the residue purified by RPHPLC (symmetry column for stationary phase and 99-90 acetonitrile/ammonium acetate mobile phase). The relevant fractions were evaporated, then azeotroped with methanol and then dried in vacuo to give the title compound as a beige oily solid (50 mg).

MS APCI+ve ^{m}/z 242 ([M+H]⁺).

¹H NMR 400 MHz (CD₃OD) 6.48 (1H, s), 6.33 (1H, s), 3.75 (1H, dd), 3.68 (2H, s), 3.21 (1H, dd), 2.91 (1H, dd), 2.21 (3H, s), 1.94 (6H, s).

### EXAMPLE 2

### S-[2-(6-Amino-4-methyl-2-pyridinyl)ethyl]-L-cysteine diacetate

### a) N-[(1,1-Dimethylethoxy)carbonyl]-S-[2-[6-(2,5-dimethyl-1H-pyrrol-1-yl)-4-methyl-2-pyridinyl]ethyl]-L-cysteine, methyl ester

To a mixture of N-(tert-butoxycarbonyl)-L-cysteine methyl ester (1.42 g), 6-(2,5-dimethyl-1H-pyrrol-1-yl)-4-methyl-2-pyridineethanol (WO 96/18616) (0.50 g), 1,1′-(azodicarbonyl)dipiperidine (1.01 g) and imidazole (0.27 g) in dry DCM (40 ml) under nitrogen, was added trimethylphosphine, (1M solution in toluene, 2 ml) dropwise is and the reaction stirred for 16 h. The reaction mixture was filtered then concentrated and the residue purified by chromatography (silica, 0-5% diethyl ether/DCM as eluent) to give the sub-titled product (0.48 g) as an oil.

MS APCI+ve ^{m}/z448 ([M+H]⁺).

¹H NMR 400 MHz (CDCl₃) some peaks split due to rotamers 6.98 (1H, s), 6.87 (1H, s), 5.87 (2H, s), 5.35 (1H, m), 4.54 (1H, m), 3.76 and 3.73 (3H, s), 3.17-2.93 (6H, m), 2.38 (3H, s), 2.11 (6H, s), 1.45 and 1.44 (9H, s).

### b) S-[2-(6-Amino-4-methyl-2-pyridinyl)ethyl]-N-[(1,1-dimethylethoxy)carbonyl-L-cysteine, methyl ester

To a solution of the product from step (a) (0.38 g) in ethanol (2.5 ml) and water (1 ml) was added hydroxylamine hydrochloride (0.27 g) and potassium hydroxide (0.15 g) and the reaction heated at 95° C. for 16 h. Water was added and the aqueous layer extracted twice with DCM. The combined organics were dried and the solvent was removed in vacuo. The residue was purified by chromatography (silica, 2-10% methanol/DCM as eluent) followed by RPHPLC (Xterra column for stationary phase and 95-50 acetonitrile/ammonia mobile phase) to give the sub-titled compound (83 mg) as a yellow oil.

¹H NMR 400 MHz (50° C., CDCl₃) 6.34 (1H, s), 6.16 (1H, s), 5.60 (1H, bs), 4.52 (1H, s), 4.32 (2H, bs), 3.74 (3H, s), 3.01-2.79 (6H, m), 2.19 (3H, s), 1.44 (9H, s).

### c) S-[2-(6-Amino-4-methyl-2-pyridinyl)ethyl]-L-cysteine diacetate

The compound from step (b) (80 mg) was treated with 5M aqueous HCl (4 ml) and heated at 95° C. for 20 min. The mixture was concentrated and the residue purified by RPHPLC (symmetry column for stationary phase and 95-80 acetonitrile/ammonium acetate mobile phase). The relevant fractions were evaporated, then azeotroped with methanol and then dried in vacuo to give the title compound as a white oily solid (25 mg).

MS APCI+ve ^{m}/z 256 ([M+H]⁺).

¹H NMR 400 MHz (CD₃OD) 6.47 (1H, s), 6.38 (1H, bs), 3.75 (1H, bs), 3.16 (1H, dd), 3.01 (1H, dd), 2.88 (4H, m), 2.23 (3H, s), 1.94 (6H, s).

### EXAMPLE 3

### S-[(6-Amino-4-methyl-2-pyridinyl)methyl]-L-homocysteine diacetate

### a) N-[(1,1-Dimethylethoxy)carbonyl]-S-[[6-(2,5-dimethyl-1H-pyrrol-1-yl)-4-methyl-2-pyridinyl]methyl]-L-homocysteine, methyl ester

To a solution of L-homocysteine sodium salt (0.31 g) in DMF (2 ml) was added sodium hydride (60% dispersion in mineral oil, 120 mg) and the reaction stirred for 2 h. A solution of the product from Example 1 step (a) (0.40 g) in DMF was added dropwise and the mixture stirred for 16 h. Water (1 ml) and di-tert-butyl dicarbonate (0.44 g) were added, and the reaction stirred for 3 h. DCM and 1M hydrochloric acid were added and the organic layer was separated, dried and the solvent was removed in vacuo. The residue was taken up in toluene/methanol (3:1, 20 ml), and trimethylsilyldiazomethane (1 M solution in hexanes, 4 ml) was added dropwise and the reaction stirred for 30 min. The volatiles were removed in vacuo and the residue was purified by chromatography (silica, 0-5% diethyl ether/DCM as eluent) to give the sub-title compound (0.25 g) as a yellow oil containing some minor impurities.

MS APCI+ve ^{m}/z 448 ([M-CH₃+H]⁺).

¹H NMR 400 MHz (CDCl₃) 7.20 (1H, s), 6.89 (1H, s), 5.86 (2H, s), 5.03 (1H, m), 4.36 (1H, m), 3.81 (2H, s), 3.69 (3H, s), 2.57 (2H, t), 2.41 (3H, s), 2.10 (6H, s), 1.91 (2H, septet), 1.43 (9H, s).

### b) S-[(6-Amino-4-methyl-2-pyridinyl)methyl]-N-[(1,1-dimethylethoxy)carbonyl]-L-homocysteine, methyl ester

Prepared from the product from step (a) using the method described in Example 1 step (c). Red oily solid.

MS APCI+ve ^{m}/z 370 ([M-(tBuOC═O)+H]⁺).

### c) S-[(6-Amino-4-methyl-2-pyridinyl)methyl]-L-homocysteine diacetate

Prepared from the product from step (b) using the method described in Example 1 step (d).

MS APCI+ve ^{m}/z 242 ([M+H]⁺).

¹H NMR 400 MHz (CD₃OD) 6.52 (1H, s), 6.35 (1H, s), 3.69 (1H, dd), 3.64 (2H, s), 2.67 (2H, t), 2.30-2.04 (2H, m), 2.24 (3H, s), 1.96 (6H, s).

### EXAMPLE 4

### S-[(6-Amino-4-methyl-2-pyridinyl)methyl]-2-methyl-L-cysteine diacetate

### a) S-[(6-Amino-4-methyl-2-pyridinyl)methyl]-2-methyl-L-cysteine diacetate

Prepared from the product from Example 1 step (a) and 2-methyl-L-cysteine hydrochloride according to the procedures described in Example 3.

MS APCI+ve ^{m}/z 256 ([M+H]⁺).

¹H NMR 400 MHz (CD₃OD) 6.36 (1H, s), 6.22 (1H, s), 3.59 (2H, q), 3.11 (1H, d), 2.76 (1H, d), 2.11 (3H, s), 1.85 (6H, s), 1.39 (3H, s).

### EXAMPLE 5

### (3R)—S-[(6-Amino-4-methyl-2-pyridinyl)methyl]-3-methyl-L-cysteine diacetate

### a) N-[(1,1-Dimethylethoxy)carbonyl]-O-[(4-methylphenyl)sulfonyl]-L-allothreonine, methyl ester

To a solution of N-[(1,1-dimethylethoxy)carbonyl]-L-allothreonine, methyl ester (5.88 g) in pyridine (73 ml) at −20° C. was added p-toluenesulfonyl chloride (48 g), and the reaction was warmed to 0° C. After 8 h, the mixture was poured onto ice (ca. 200 g) and extracted twice with ethyl acetate. The combined organics were washed with water, brine, 10% aqueous citric acid solution (2×), aqueous sodium bicarbonate solution (2×), brine, dried and the solvent was removed in vacuo. The residue purified by chromatography (silica, toluene then ethyl acetate as eluents) to give the sub-title compound (5.84 g) as a yellow oil.

MS APCI+ve ^{m}/z 388 ([M+H]⁺)

### b) (3R)—S-Benzoyl-N-[(1,1-dimethylethoxy)carbonyl]-3-methyl-L-cysteine, methyl ester

To solid potassium thiobenzoate (5 g) at 0° C. was added a solution of the product from step (a) (2.5 g) in cold DMP (10 ml) and the reaction warmed to room temperature. After 16 h, the reaction was diluted with ethyl acetate and washed with water (2×), brine, aqueous sodium bicarbonate solution, brine, dried and the solvent was removed in vacuo to give the sub-title compound (1.94 g) as an yellow oil.

MS APCI+ve ^{m}/z 254 ([M-(tBuOC═O)+H]⁺).

### c) (3R)-N-[(1,1-Dimethylethoxy)carbonyl]-S-[[6-(2,5-dimethyl-1H-pyrrol-1-yl)-4-methyl-2-pyridinyl]methyl]-3-methyl-L-cysteine, methyl ester

To a solution of the product from step (b) in methanol (3 ml) at 0° C. was added sodium methoxide (0.11 g). After 2 h, a solution of the product from Example 1 step (a) (0.47 g) in acetonitrile (3 ml) was added dropwise and the reaction warmed to room temperature and then heated at 55° C. for 5 h. After cooling to room temperature, the volatiles were removed in vacuo. Diethyl ether was added, and the organic solution washed with water (3×), aqueous sodium bicarbonate solution, 10% aqueous citric acid solution, brine, dried and the solvent was removed in vacuo. The residue purified by chromatography (silica, 0-12% diethyl ether/DCM as eluent) to give the sub-title compound (0.33 g) as a colourless oil.

MS APCI+ve ^{m}/z 448 ([M+H]⁺).

### d) (3R)—S-[(6-Amino-4-methyl-2-pyridinyl)methyl1-3-methyl-L-cysteine diacetate

Prepared from the compound from step (c) according to the procedures described in Example 1 steps (c) and (d) to give the title compound as a beige solid.

MS APCI+ve ^{m}/z 256 ([M+H]⁺).

¹H NMR 400 MHz (CD₃OD) 6.49 (1H, s), 6.32 (1H, s), 3.75 (1H, d), 3.64 (1H, d), 3.54 (1H, d), 3.40 (1H, quintet), 2.20 (3H, s), 1.93 (6H, s), 1.42 (3H, d).

### EXAMPLE 6

### O-[(6-Amino-4-methyl-2-pyridinyl)methyl]-L-serine acetate

### a) N-[(1,1-Dimethylethoxy)carbonyl]-O-[[6-(2,5-dimethyl-1H-pyrrol-1-yl)-4-methyl-2-pyridinyl]methyl]-L-serine, methyl ester

To a solution of N-(tert-butoxycarbonyl)-L-serine (0.30 g) in DMP (5 ml) was added sodium hydride (60% dispersion in mineral oil, 58 mg) and the reaction stirred for 1 h. A solution of the product from Example 1 step (a) (0.38 g) in acetonitrile (4 ml) was added dropwise and the mixture stirred for 16 h. Ethyl acetate and water were added and the aqueous layer separated, acidified with 2M aqueous HCl and then extracted with DCM. The organic layer was dried and the solvent was removed in vacuo to give a brown oil. This oil was dissolved in toluene (15 ml) and water (5 ml) and treated with (trimethylsilyl)diazomethane (2M solution in hexane, 1.6 ml) and stirred for 1 h. The solvent was removed in vacuo to give the sub-titled compound (0.51 g) as a brown oil.

MS APCI+ve ^{m}/z 418 ([M+H]⁺).

### b) O-[(6-Amino-4-methyl-2-pyridinyl)methyl]-N-[(1,1-dimethylethoxy)carbonyl]-L-serine, methyl ester

To a solution of the product from step (a) (0.51 g) in ethanol (3 ml) and water (1 ml) was added hydroxylamine hydrochloride (0.38 g) and potassium hydroxide (0.21 g) and the reaction heated at 95° C. for 16 h. Water was added and the aqueous layer extracted with DCM (3×). The combined organics were dried and the solvent was removed in vacuo. The residue was passed through a sinter pad of silica and eluted with DCM and then the solvent was removed in vacuo to give the sub-titled compound as an oil.

MS APCI+ve ^{m}/z ₃₄₀ ([M+H]⁺).

### c) O-[(6-Amino-4-methyl-2-pyridinyl)methyl]-L-serine acetate

The compound from step (b) was treated with 5 M aqueous HCl (2 ml) and heated at 95° C. for 1 h. The mixture was concentrated and the residue purified by RPHPLC (symmetry column for stationary phase and 95-80 acetonitrile/ammonium acetate mobile phase). The relevant fractions were evaporated, then azeotroped with methanol and then dried in vacuo to give the title compound as a white solid (2 mg).

MS APCI+ve ^{m}/z ₂₂₆ ([M+H]⁺).

¹H NMR 400 MHz (CD₃OD) 6.57 (1H, s), 6.54 (1H, s), 4.50 (2H, q), 3.87 (2H, m), 3.75 (1H, m), 2.60 (3H, s), 2.24 (3H, s).

### EXAMPLE 7

### O-[(6-Amino-4-methyl-2-pyridinyl)methyl]-D-serine

### a) N-[(1,1-Dimethylethoxy)carbonyl]-O-[[6-(2,5-dimethyl-1H-pyrrol-1-yl)-4-methyl-2-pyridinyl]methyl]-D-serine methyl ester

The sub-titled compound was prepared as for Example 6 step (a) using N-(tert-butoxycarbonyl)-D-serine (0.35 g) to give the compound as a brown oil.

MS APCI+ve ^{m}/z 418 ([M+H]⁺).

### b) O-[(6-Amino-4-methyl-2-pyridinyl)methyl]-N-[(1,1-dimethylethoxy)carbonyl]-D-serine, methyl ester

To a solution of the product from step (a) (0.6 g) in ethanol (4 ml) and water (1.5 ml) were added hydroxylamine hydrochloride (0.44 g) and potassium hydroxide (0.24 g) and the reaction heated at 95° C. for 16 h. Water was added and the aqueous layer extracted with DCM (3×). The combined organics were dried and the solvent was removed in vacuo. The solvent was removed in vacuo to give the sub-titled compound as an oil.

MS APCI+ve ^{m}/z 340 ([M+H]⁺).

### c) O-[(6-Amino-4-methyl-2-pyridinyl)methyl]-D-serine

The compound from step (b) was treated with 5M aqueous HCl (4 11l) and heated at 95° C. for 1 h. The mixture was concentrated and the residue purified by RPHPLC (symmetry column for stationary phase and 95-85 acetonitrile/ammonium acetate mobile phase). The relevant fractions were evaporated and azeotroped with toluene (3×), then ether (2×), to give a clear oil. The oil was dissolved in a minimum volume of methanol and precipitated with ether (5 ml), then dried in vacuo to give the title compound as a white solid (20 mg).

MS APCI+ve ^{m}/z 226 ([M+H]⁺),

¹H NMR 400 MHz (CD₃OD) 6.50 (1H, s), 6.38 (1H, s), 4.41 (2H, q), 3.83 (2H, m), 3.70 (1H, m), 2.18 (3H, s).

### EXAMPLE 8

### 3-[[(6-Amino-4-methyl-2-pyridinyl)methyl](methylsulfonyl)amino]-L-alanine dihydrochloride

### a) 2-Bromo-6-(2,5-dimethyl-1H-pyrrol-1-yl)-4-methyl-pyridine

Acetonylacetone (14.7 ml), sodium acetate (100 mg) and acetic acid (1 ml) were added to a solution of 2-bromo4-methyl-6-aminopyridine (J. Org. Chem., 1962, 27, 2473) (15 g) in toluene (100 ml). The reaction mixture was heated under vigorous reflux for 48 h, the water produced being removed using a Dean-Stark apparatus. The reaction mixture was allowed to cool to room temperature and was then diluted with ethyl acetate. The solution was then washed with 2.5M sodium hydroxide solution, water and brine, then dried over MgSO₄ and evaporated to dryness. The residue was purified by chromatography (silica, 10% ethyl acetate/isohexane as eluent) to give the sub-titled product as a yellow solid (17.5 g).

MS APCI+ve ^{m}/z 265/267 ([M+H]⁺).

### b) 6-(2,5-Dimethyl-1H-pyrrol-1-yl)-4-methyl-2-pyridinecarboxaldehyde

The product from step (a) (1.33 g) was dissolved in dry THF and cooled to −78° C. under an atmosphere of nitrogen. A solution of n-butyl lithium (1.6M solution in hexanes, 3.75 ml) was added whilst maintaining the temperature below −65° C. The reaction mixture was stirred at this temperature for 10 min before DMF (0.47 ml) was added dropwise, maintaining the temperature below −65° C. The reaction mixture was then stirred for a further 2 h. Saturated ammonium chloride solution was then added and the reaction mixture was extracted with ethyl acetate. The organic phase was dried over MgSO₄, filtered and evaporated to dryness. The residue was purified by chromatography (silica, 10% ethyl acetate/isohexane as eluent) to give the sub-titled compound as a yellow solid (0.7 g).

MS APCI+ve ^{m}/z 215 ([M+H]⁺).

### c) 3-[[[6-(2,5-Dimethyl-1H-pyrrol-1-yl)-4-methyl-2-pyridinyl]methyl]amino]-N-[(phenylmethoxy)carbonyl]-)-L-alanine, methyl ester

The product from step (b) (0.7 g), (S)-methyl-3-amino-2-benzyloxycarbonylaminopropionate hydrochloride (J. Med. Chem., 41, 2786-2805) (0.94 g) and triethylamine (0.9 ml) were suspended in 1,2-dichloroethane (20 ml). Sodium triacetoxyborohydride (1.02 g) was then added and the reaction mixture was stirred at room temperature under an atmosphere of nitrogen for 72 h. The reaction mixture was then quenched with saturated sodium bicarbonate solution and extracted with ethyl acetate. The organic phase was dried over MgSO₄, filtered and the filtrate was evaporated to dryness. The residue was purified by chromatography (silica, ethyl acetate as eluent) to give the sub-titled compound as a colourless oil (1.0 g).

MS APCI+ve ^{m}/z 451 ([M+H]⁺).

### d) 3-[[[6-(2,5-Dimethyl-1H-pyrrol-1-yl)-4-methylpyridinyl]methyl]-(methylsulfonyl)amino]-N-[(phenylmethoxy)carbonyl]-L-alanine, methyl ester

A solution of the product from step (c) (0.5 g), triethylamine (0.17 ml), methanesulfonyl chloride (0.10 ml) and N,N-dimethylaminopyridine in DCM (10 ml) was stirred at room temperature for 2.5 h. Further portions of triethylamine (0.17 ml) and methanesulfonyl chloride (0.10 ml) were added and the reaction mixture was stirred at room temperature for 3 h. The reaction mixture was then washed with water, saturated sodium carbonate solution and brine. The organic phase was dried over MgSO₄, filtered and evaporated. The residue was purified by chromatography (silica, ethyl acetate as eluent) to give the sub-titled compound as a yellow oil (0.54 g).

MS APCI+ve ^{m}/z 529 ([M+H]⁺).

### e) 3-[[(6-Amino-4-methyl-2-pyridinyl)methyl](methylsulfonyl)amino]-N-[(phenylmethoxy)carbonyl]-L-alanine

A suspension of the product from step (d) (0.534 g), hydroxylamine hydrochloride (0.32 g) and potassium hydroxide (0.178 g) in water (1.5 ml) and ethanol (3.0 ml) was heated under reflux for 4 h. On cooling to room temperature the mixture was poured onto saturated ammonium chloride solution and extracted with DCM. The organic phase was dried over MgSO₄, filtered and evaporated. The residue was purified by chromatography (silica, 33% ethyl acetate/DCM as eluent) to give the subtitle compound as an off-white solid (0.12 g).

MS APCI+ve ^{m}/z 437 ([M+H]⁺).

### f) 3-[[(6-Amino-4-methyl-2-pyridinyl)methyl](methylsulfonyl)amino]-)-L-alanine

A suspension of the product from step (e) (97 mg) and 10% palladium on charcoal (15 mg) in ethanol (5 ml) was stirred under an atmosphere of hydrogen for 16 h. The catalyst was filtered off and the filtrate was evaporated to dryness. The residue was triturated with 1M hydrogen chloride in diethyl ether followed by extensive trituration with ethyl acetate to give the title compound as an off-white solid (24 mg).

MS APCI+ve ^{m}/z 303 ([M+H]⁺).

¹H NMR 400 MHz (CD₃OD) 6.51 (1H, s), 6.46 (1H, s), 4.36 (2H, q), 3.77 (2H, m), 3.75 (1H, m), 2.95 (3H, s), 2.21 (3H, s).

### EXAMPLE 9

### 3-[[(6-Amino-4-methyl-2-pyridinyl)methyl]amino]-L-alanine trihydrochloride

### a) Methyl N-[(1,1-dimethylethoxy)carbonyl]-3-[[1,1-dimethylethoxy)carbonyl][[6-(2,5-dimethyl-1H-pyrrol-1-yl)-4-methyl-2-pyridinyl]methyl]amino]-L-alanine

The product from Example 8 step (b) (1.07 g), methyl, 3-amino-N-[(1,1-dimethylethoxy)carbonyl]-L-alanine (1.02 g) and triethylamine (0.70 ml) were suspended in 1,2-dichloroethane (30 ml). Sodium triacetoxyborohydride (1.48 g) was then added and the reaction mixture was stirred at room temperature under an atmosphere of nitrogen for 16 h. The reaction mixture was then quenched with saturated sodium bicarbonate solution and the organic phase was separated. Di-tert-butyl dicarbonate (2.0 g) was added to the aqueous phase and the reaction mixture was then stirred at room temperature for 16 h. The reaction mixture was then washed with DCM before acidifying the aqueous phase with citric acid solution. The aqueous phase was then extracted into DCM. The organic phase was dried over MgSO₄, filtered and evaporated. Trimethylsilyldiazomethane (2M solution in hexanes, 4.0 ml) was then added to a solution of the residue in methanol (10 ml) and toluene (35 ml). The reaction mixture was then stirred at room temperature for 4 h. The solution was evaporated to dryness and the residue was purified by chromatography (silica, 20% ethyl acetate/DCM as eluent) to give the sub-titled compound as an off-white solid (1.0 g).

MS APCI+ve ^{m}/z 517 ([M+H]⁺).

### b) Methyl 3-[[(6-amino-4-methyl-2-pyridinyl)methyl][(1,1-dimethylethoxy)carbonyl]amino]-N-[(1,1-dimethylethoxy)carbonyl]-L-alanine

Prepared from the product from step (a) according to the method described in Example 8 step (e).

MS APCI+ve ^{m}/z 439 ([M+H]⁺).

### c) 3-[[(6-Amino-4-methyl-2-pyridinyl)methyl]amino]-L-alanine

A suspension of the product from step (b) (0.52 g) in 6M hydrochloric acid (20 ml) was heated under reflux for 4 h. The solution was evaporated to dryness and the residue was re-dissolved in water (20 ml). This solution was then freeze dried for 16 h. The residue was 30 then triturated with diethyl ether to give the title compound as an off-white solid (0.2 g).

MS APCI+ve ^{m}/z 225 ([M+H]⁺).

¹H NMR 400 MHz (D₂O) 6.93 (1H, s), 6.86 (1H, s), 4.45 (2H, m), 4.17 (1H, m), 3.61 (2H, m), 2.42 (3H, s).

### EXAMPLE 10

### (3S)-S-[(6-Amino-4-methyl-2-pyridinyl)methyl]-3-methyl-L-cysteine monoacetate

### a) 6-(2,5-Dimethyl-1H-pyrrol-1-yl)-4-methyl-2-pyridinemethanol

To a solution of the product from Example 8 step (a) (4.0 g) in THF (60 ml), at −75° C. under a nitrogen atmosphere, was added n-butyl lithium (1.6M solution in hexanes, 11.3 ml) dropwise. The solution was allowed to warm to 0° C. over 1 h and then formaldehyde gas in nitrogen (prepared by heating paraformaldehyde (9 g) at 160° C.) was bubbled through the solution, via a cannula. After 1 h, the reaction was diluted with ethyl acetate, washed twice with water, dried and the solvent was removed in vacuo to give an orange oil. The residue was purified by chromatography (silica, ethyl acetate/isohexane as eluents) to give the sub-title compound (0.6 g) as an oil.

MS APCI+ve ^{m}/z 217 ([M+H]⁺).

¹H NMR 400 MHz (CDCl₃) 7.07 (1H, s), 6.94 (1H, s), 5.91 (2H, s), 4.75 (2H, s), 2.44 (3H, s), 2.13 (6H, s).

### b) 6-Amino-4-methylpyridin-2-ylmethanol

To a solution of the product from step (a) (0.6 g) in ethanol (4 ml) and water (1.0 ml) was added hydroxylamine hydrochloride (0.87 g) and potassium hydroxide (0.53 g) and the reaction heated at 95° C. for 16 h. After cooling to room temperature, the reaction was diluted with DCM and extracted with 2M hydrochloric acid. The aqueous layer was then basified using potassium hydroxide and then extracted with DCM (6×). The combined organics were dried and the solvent removed in vacuo to give the sub-titled compound (0.36 g) as an off white solid.

MS APCI+ve ^{m}/z 1₃₉ ([M+H ⁺).

¹H N 300 MHz (CDCl₃) 6.42 (1H, s), 6.22 (1H, s), 4.55 (2H, s), 4.36 (2H, s) 2.23 (3H, s).

### c) 6-(Chloromethyl)4-methyl-2-pyridinamine hydrochloride

The compound from step (b) was treated with excess thionyl chloride and stirred at room temperature for 1 h. The solvent was then removed in vacuo and the resulting gum was triturated with DCM to give the sub-titled compound (0.44 g) as a yellow solid.

MS APCI+ve ^{m}/z 157 ([M+H]⁺).

¹H NMR 400 MHz (DMSO-d₆) 6.83 (1H, s), 6.73 (1H, s), 4.77 (2H, s), 2.32 (3H, s).

### d) N-[(1,1-Dimethylethoxy)carbonyl]-O-[(4-methylphenyl)sulfonyl]-L-threonine methyl ester

The sub-titled compound was prepared by the method of Example 5 step (a) using N-[(1,1-dimethylethoxy)carbonyl]-L-threonine methyl ester (3.5 g) to give the crude product as an oily yellow solid (7.6 g, 50% pure).

MS APCI+ve ^{m}/z 288 ([M-(tBuOC═O)+H] ).

### e) (3S)-S-Benzoyl-N-[(1,1-dimethylethoxy)carbonyl]-3-methyl-L-cysteine methyl ester

The sub-titled compound was prepared by the method of Example 5 step (b) using the product from step (d) above (7.6 g) to give the crude product as a yellow oil (3.8 g, 85% pure).

MS APCI+ve ^{m}/z 254 ([M-(tBuOC═O)+H]⁺).

### f) (3S)-S-[(6-Amino-4-methyl-2-pyridinyl)methyl]-3-methyl-L-cysteine monoacetate

The product from step (e) (388 mg) was treated with sodium methoxide (54 mg) in methanol (3 ml) at 0° C. A solution of the compound from step (c) (154 mg) in methanol (2 ml) was added dropwise, then the mixture was warmed up to ambient temperature and stirred for 1 h. The solution was then heated to 55° C. and stirred for a further 12 h after which more sodium methoxide (54 mg) was added and stirred for 30 min. The volatiles were removed in vacuo and the resulting gum was extracted into ether and washed twice with water, aqueous sodium bicarbonate solution, aqueous citric acid, brine and the solvent was removed in vacuo. The resulting residue was then treated with 5M hydrochloric acid (4 ml) and heated at 95° C. for 2 h. The mixture was concentrated and the residue purified by RPHPLC (symmetry C18 column for stationary phase and 95-50 methanol/ammonium acetate mobile phase). The relevant fractions were evaporated and azeotroped with toluene (3×), then ether (2×), to give the title compound as a white solid (60 mg).

MS APCI+ve ^{m}/z 256 ([M+H]⁺).

¹H NMR 300 MHz (CD₃OD) 6.52 (1H, s), 6.34 (1H, s), 3.90 (1H, d), 3.80-3.61 (3H, m), 2.23 (3H, s), 2.23 (4H, s), 1.25 (3H, d).

### Screens

The pharmacological activity of compounds according to the invention was tested in the following screens.

Screen 1

Recombinant human NO synthases (iNOS, eNOS & nNOS) were expressed in E. coli and lysates were prepared in Hepes buffer (pH 7.4) containing co-factors (FAD, FMN, H₄B), protease inhibitors, lysozyme and the detergent, CHAPS. These preparations were used, at suitable dilution, to assess inhibition of the various isoforms. Inhibition of NOS was determined by measuring the formation of L-[³H]citrulline from L-[³H]arginine using an adaptation of the method of Förstermann et al.⁹ Enzyme assays were performed in the presence of 3 μM [³H]arginine, 1 mM NADPH and other co-factors required to support NOS activity (FAD, FMN, H₄B, calmodulin, Ca²⁺). Since various NOS inhibitors have been reported to exhibit slow binding kinetics, or to inactivate the enzyme in a time dependent manner, enzyme and inhibitor were pre-incubated for 60 min in the presence of NADPH before addition of arginine to initiate the reaction. Incubations continued for a further 60 min before the assays were quenched and [³H]citrulline separated from unreacted substrate by chromatography on Dowex-50W resin in a 96-well format.

In the above screen, the compounds of Examples 1 to 10 were tested and gave IC₅₀ values of less than 10 μM against the iNOS enzyme indicating that they are expected to show useful therapeutic activity.

Screen 2

Compounds also show activity against the human form of induced nitric oxide synthase as can be demonstrated in the following assay.

The human colorectal carcinoma cell line, DLD-1 (obtained from the European Collection of Animal Cell Culture—cell line number 90102540) was routinely grown in RPMI 1640 supplemented with 10%(v/v) foetal bovine serum, and 2mM L-glutamine, at 37° C. in 5% CO₂.

Nitric oxide synthase was induced in cells by addition of medium containing human recombinant gamma-IFN (1000 units/ml), TNF-alpha (200 U/ml), IL-6 (200 U/ml) and IL-1-beta (250 U/ml). After incubation for 18 hours at 37° C., the medium was removed and the cells washed with warm phosphate buffered saline. Cells were incubated for a further 5 hours at 37° C./5% CO₂ in RPMI 1640 containing 100 μM L-arginine and 100 μM verapamil-HCl in the presence and absence of test compounds.

Nitrite accumulation was determined by mixing an equal volume of culture media with Griess reagent (10 mg/ml sulphanilamide, 1 mg N-(1-naphthyl)ethylenediamine in 1 ml 2.5% (v/v) phosphoric acid). Inhibition in the presence of compounds was calculated relative to the nitrite levels produced by untreated cells. IC₅₀ values were estimated from a semi-log plot of % inhibition versus concentration of compound.

In this screen the compounds of Examples 1 to 9 gave IC₅₀ values of less than 100 μM, indicating that they are predicted to show useful therapeutic activity.

1. A compound of formula (I) 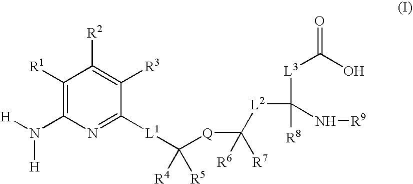
wherein
R¹, R² and R³ independently represent H, halogen, C1to 4 alkyl, C1to 4 alkoxy, CN, MeS(O)ₘ or NR¹⁰R¹¹; said alkyl group being optionally further substituted by OH or one or more halogen atoms;
L¹ and L² independently represent a bond or CR¹²R¹³ wherein R¹² and R¹³ independently represent H or C1to 4 alkyl; said alkyl being optionally further substituted by OH, C1to 2 alkoxy, CN or one or more halogen atoms;
L³ represents —CH₂— or a bond;
R⁴, R⁵, R⁶ and R⁷ independently represent H, C1to 6 alkyl, Ar¹ or Ar¹—C1 to 4 alkyl;
or R⁴ and R⁵, or R⁶ and R⁷, may be joined together such that the group CR⁴R⁵ or the group CR⁶R⁷ represents a C3 to 6 cycloalkyl ring;
Q represents O, S(O)ₙ or NR¹⁶;
R¹⁶ represents H, C1 to 6 alkyl, C1 to 6 alkanoyl, C1 to 6 alkyl-SO₂—,
C1 to 6 alkyl-O—CO—, Ar² or Ar²—CH₂—;
Ar¹ and Ar² independently represents phenyl or a 5- or 6-membered heteroaromatic ring containing one to three heteroatoms independently selected from O, S and N; said phenyl or heteroaromatic ring being optionally substituted by one or more substituents independently selected from halogen, CN, CF₃, C1to 3 alkyl, C1to 3 alkoxy, hydroxy, C1to 3 thioalkoxy or NR¹⁴R¹⁵;
m and n independently represent an integer 0, 1 or 2;
R⁸ represents H or C1to 4 alkyl; said alkyl being optionally further substituted by OH, C1to 2 alkoxy, CN or one or more halogen atoms;
R⁹ represents H or C1 to 4 alkyl;
R¹⁰ and R¹¹ independently represent H, C1to 2 alkyl, C1to 2 alkanoyl or C1to 2 alkylsulfonyl;
R¹⁴ and R¹⁵ independently represent H, C1to 4 alkyl, C1to 2 alkylsulfonyl or C1 to 4 alkanoyl;
said alkyl being optionally further substituted by OH, C1to 2 alkoxy, CN or one or more halogen atoms;
and pharmaceutically acceptable salts thereof

2. A compound according to claim 1 wherein Q represents S.

3. A compound of formula (I), according to claim 1, which is:
S-[(6-amino-4-methyl-2-pyridinyl)methyl]-L-cysteine;
S-[2-(6-amino-4-methyl-2-pyridinyl)ethyl]-L-cysteine;
S-[(6-amino-4-methyl-2-pyridinyl)methyl]-L-homocysteine;
S-[(6-amino-4-methyl-2-pyridinyl)methyl]-2-methyl-L-cysteine;
(3R)-S-[(6-amino-4-methyl-2-pyridinyl)methyl]-3-methyl-L-cysteine;
O-[(6-amino-4-methyl-2-pyridinyl)methyl]-L-serine;
O-[(6-amino-4-methyl-2-pyridinyl)methyl]-D-serine;
3-[[(6-amino-4-methyl-2-pyridinyl)methyl](methylsulfonyl)amino]-L-alanine;
3-[[(6-amino-4-methyl-2-pyridinyl)methyl]amino]-L-alanine;
(3S)-S-[(6-amino-4-methyl-2-pyridinyl)methyl]-3-methyl-L-cysteine;
or a pharmaceutically acceptable salt thereof.

4. (canceled)

5. A pharmaceutical composition comprising a compound of formula (I) according to claim 1, or a pharmaceutically acceptable salt thereof, in admixture with a pharmaceutically acceptable adjuvant, diluent or carrier.

6-12. (canceled)

13. A method of treating, or reducing the risk of, a human disease or condition in which inhibition of nitric oxide synthase activity is beneficial which comprises administering a therapeutically effective amount of a compound of formula (I), as defined in claim 1, or a pharmaceutically acceptable salt thereof, to a person suffering from, or at increased risk of, said disease or condition.

14. A method of treating, or reducing the risk of, inflammatory disease in a person suffering from, or at risk of, said disease, wherein the method comprises administering to the person a therapeutically effective amount of a compound of formula (I), as defined in claim 1, or a pharmaceutically acceptable salt thereof.

15. A process for the preparation of a first compound of formula (I), as defined in claim 1, or a pharmaceutically acceptable salt, enantiomer or racemate thereof, wherein the process comprises:
(a) reaction of a compound of formula (II) 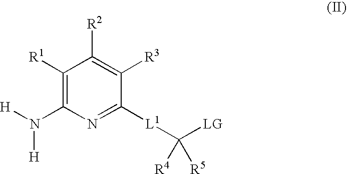
wherein LG represents a leaving group,
with a compound of formula (III) 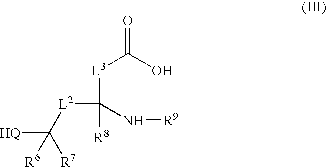
or
(b) reaction of a compound of formula (IV) 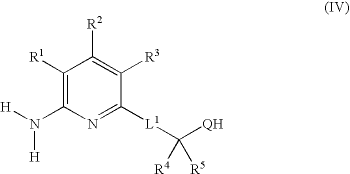
with a compound of formula (V) 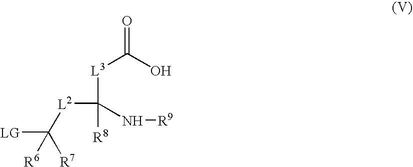
wherein LG is a leaving group; or
(c) when Q represents S, reacting a compound of formula (VI) 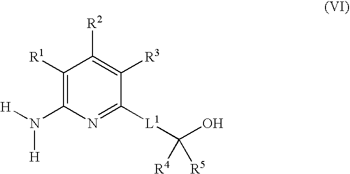
with a compound of formula (VII) 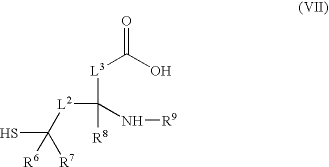
under Mitsunobu conditions;
wherein the variable groups shown above are, unless otherwise specified, as defined in claim 1; and where desired or necessary converting the first compound of formula (I), or another salt thereof, into a pharmaceutically acceptable salt thereof; or converting the first compound of formula (I) into a second compound of formula (I); and where desired converting the first compound of formula (I) into an optical isomer thereof.

16. The method as claimed in claim 13, wherein it is predominantly inducible nitric oxide synthase that is inhibited.

17. The method as claimed in claim 14, wherein the disease is rheumatoid arthritis.

18. The method as claimed in claim 14, wherein the disease is osteoarthritis.

19. A method for the treatment or prophylaxis of pain, comprising administering a therapeutically effective amount of a compound of formula (I), as defined in claim 1, or a pharmaceutically acceptable salt thereof.

20. A method for the treatment or prophylaxis of inflammatory disease, comprising administering a therapeutically effective amount of a compound of formula (I) as defined in claim 1, or a pharmaceutically acceptable salt thereof, in combination with a COX-2 inhibitor.
